(19) United States
(12) Patent Application Publication
(10) Pub. No.: US 2005/0059964 A1
(43) Pub. Date: Mar. 17, 2005

(54) Enhancing the effectiveness of medial branch nerve root RF neurotomy

(76) Inventor: William R. Fitz, Columbus, OH (US)

Correspondence Address: JOHN G. POSA;Gifford, Krass, Groh, Sprinkle,, Anderson & Citkowski, P.C., 280 N. Old Woodward Ave. Suite 400, Birmingham, MI, 48009-5394, US

(21) Appl. No.: 10/660,933

(22) Filed: Sep. 12, 2003

(51) Int. Cl. A61B 18/14

(52) U.S. Cl. 606/41

(57) ABSTRACT

Improved-coverage RF neurotomy instrumentation includes an introducer used to deploy a set of electrodes to an area to be treated. The introducer features a plurality of elongated, co-extensive cannula, each including an insulated, electrically conductive electrode. Each electrode has a proximal end configured for attachment to a source of energy, and an exposed distal tip to deliver the energy to a localized region, and each electrode slides within its respective cannula so as to enhance the energy coverage area.

### FIELD OF THE INVENTION

This invention relates generally to RF neurotomy and, in particular, to improving the coverage of the procedure.

### BACKGROUND OF THE INVENTION

Radio frequency (RF) neurotomy (also called RF rhizotomy or RF lesioning, depending upon the application), is a therapeutic procedure used to interrupt nociceptive pathways in patients with neck/back pain and chronic headache. The process can be used on any area of the spine, be it cervical, thoracic, or lumbar. The procedure involves the use of a needle to place a small electrode adjacent to the facet under x-ray guidance. An electric current is then used to cauterize the sensory nerves that innervate the facet joint(s). If successful, the pain relief following an RF procedure can last considerably longer than relief following local aesthetic and steroid blocks.

Various instruments have been designed in support of this procedure. In U.S. Pat. No. 4,411,266, for example, a radio frequency (RF) lesion electrode is described having a thermocouple temperature sensor in its distal uninsulated tip. The design also includes other features which enable it to be made with very small tip diameters, flexible tip geometrics, very close proximity of the thermocouple sensor to the tissue the temperature of which must be measured, and very accurate and rapid temperature response.

A problem with current designs is that they do not allow for adequate coverage of the target area. In terms of anatomy, a straight needle is generally used to cover a small nerve on a rounded bony surface. As shown in FIG. 1, a spherical lesion is produced through the heat generated at the tip of the needle, but this extends only a few millimeters at the diameter of the midportion of the lesion. Worst, if the tip is over 2 mm away from the nerve (which cannot be directly visualized), then it is not lesioned at all. Anatomy differs from patient to patient, and the target medial branch may not be in the same place with respect to different individuals. In the cervical and thoracic spine in particular, the ideal target is known to lie in variable locations.

Although multiple lesions may be created in order to enhance coverage, this is more time-consuming and usually more painful to the patient. The creation of multiple lesions also exposes the patient to more radiation, as fluoroscopy is used to place and replace the needles. In the cervical spine, six separate needle locations are at times used to obtain adequate coverage of one medial branch nerve. Since at least two medial branches are treated, this can extend the length of the procedure to three or four hours.

Other inventions have been disclosed to improve electrode coverage, though not necessarily for the spine. In accordance with the teachings of U.S. Pat. No. 5,807,395, the infusion of conducting fluid into the area of ablation or hyperthermia prior to and during the application of RF energy creates what is referred to herein as a “virtual electrode,” the size and shape of which can be controllably modified, and which can be rendered more or less conductive, thereby modifying the spread of RF energy. By varying such factors as the RF energy and duration, the extent of pre-RF infusion, the RF infusion rate and conductivity of solution, the electrode size, shape, and surface area, the size, shape, and intensity of the ”virtual electrode”—i.e., the intensity of thermal production in the ablation or hyperthermia area, can be controlled.

In U.S Pat. No. 6,442,435, an implantable lead is provided with at least one extendable member to position therapy delivery elements, which may be electrodes or drug delivery ports, after the lead has been inserted into the body. The lead may formed as a resilient element which is contained in a retainer tube that may be removed to permit the lead to deploy. Alternatively, a non-resilient lead may be provided with a slotted retainer tube. A series of mechanical linkages for expanding and retracting the lead within the human body may be actuated with various mechanisms. A control system may be provided for closed-loop feedback control of the position of the extendable members. The invention also includes a method for expanding an implantable lead in situ.

Existing designs have various shortcomings. For example, some require an open space such as the spinal canal, and are not readily deployable through muscle tissue. Other designs, like the one described above, require side ports for an aesthetic administration. Overall, due to different deficiencies, current methods do not provide adequate coverage of the treated area.

### SUMMARY OF THE INVENTION

This invention resides in improved-coverage RF neurotomy instrumentation, including an introducer used to deploy a set of electrodes to an area to be treated. The introducer features a plurality of elongated, co-extensive cannula, each including an insulated, electrically conductive electrode. Each electrode has a proximal end configured for attachment to a source of energy, and an exposed distal tip to deliver the energy to a localized region, and each electrode slides within its respective cannula so as to enhance the energy coverage area.

In the preferred embodiment, the distal tips of at least some of the electrodes are beveled to facilitate navigation. Alternatively, at least some of the electrodes are constructed of a shape-memory material to control deployment. The cannula are generally parallel, and may either lie in the same plane or may be arranged otherwise, including spoke-like cross-sections.

The introducer may be straight or curved, and at least one side port may be provided for the administration of an anethestic. At least some of the electrodes may slide independently, and at least some of the electrodes may slide in unison.

### BRIEF DESCRIPTION OF THE DRAWINGS

FIG. 1 is a drawing of a prior art needle tip, showing the limited area of coverage;

FIG. 2 is a drawing of an instrument according to the invention, including a parallel introducer with electrodes projecting from the introducer tip;

FIG. 3 is a drawing of an alternative embodiment of the invention in the form of a quad introducer;

FIG. 4 is a drawing of yet a further alternative embodiment of the invention in the form of a side-port introducer; and

FIG. 5 is a drawing of yet a further alternative embodiment of the invention in the form of a radial array, which may introduce from the tip, or some distance from the tip.

### DETAILED DESCRIPTION OF THE INVENTION

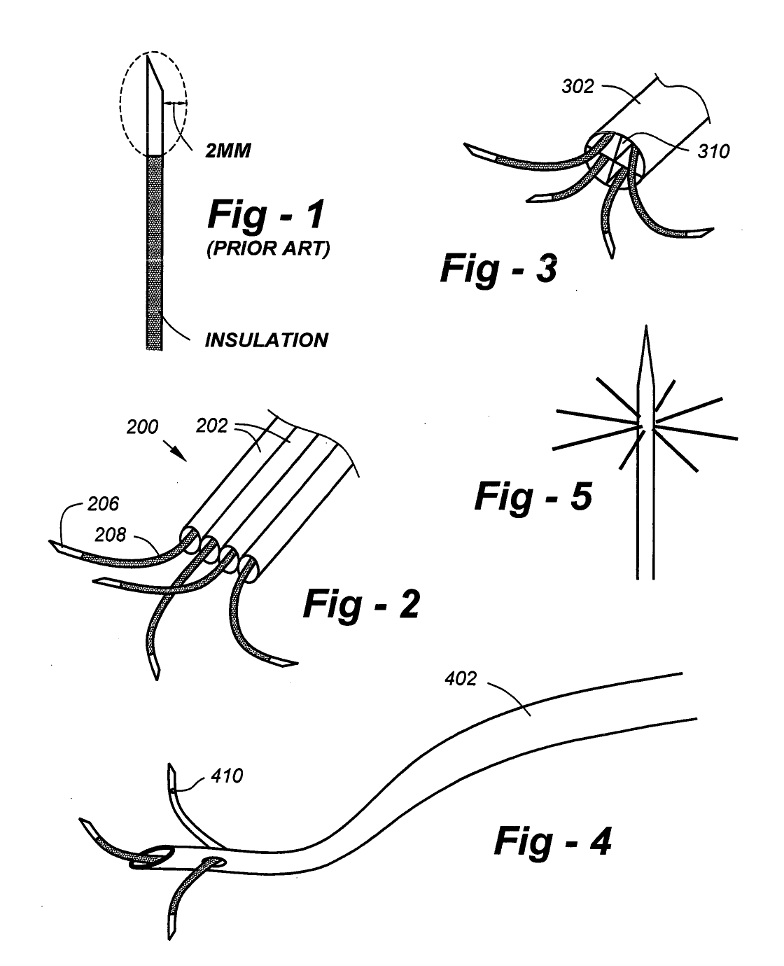

Having described the problems associated with the prior-art arrangement of FIG. 1, reference is now made to FIG. 2, which shows one embodiment of the invention in the form of a parallel series introducer, depicted generally at 200. According to this embodiment, a plurality of side-by-side cannula 202 are provided, each with an electrode such as 206 projecting therefrom for individual or collective deployment. The cannula 202 may be constructed from any suitable material, including metal and plastics/polymers, though in the preferred embodiment, thin-walled metal is used for lightweight rigidity.

This and in other embodiments, the tips of the electrodes are beveled for directional control. Shape-memory materials may also be used to enhance directionality. It is noted that energy supply and control electronics used in conjunction with the electrodes may take advantage of any appropriate technology, and do not necessarily constitute a point of novelty with respect to this invention. That is, any suitable power supply and/or control electronics may be used. The electrodes include lengthwise insulation 208, but for the exposed distal tip and connection to external power-supply and control electronics (not shown).

FIG. 3 illustrates an alternative embodiment of the invention, in the form of a cannulated sleeve 302 having a central longitudinal separator 310, again allowing the electrodes to be independently or collectively slidingly received therein. In the preferred configuration, a cruciate separator is used and, the electrodes again include beveled tips and/or are constructed from shape-memory material to enhance navigation.

FIG. 4 illustrates yet a further alternative embodiment of the invention, particularly applicable to side port introduction. In this case, the introducer 402 is curved, and a plurality of electrodes are deployed through the tip and/or multiple side ports. In this and the other designs disclosed herein, the sideport(s) 410 may be provided for an aesthetic administration, with or without electrical insulation.

Turning now to FIG. 5, yet a further alternative embodiment of the invention is depicted, in this case utilizing an introducer having a tip and sufficient side ports to form an array of electrodes once deployed. Although the side electrodes are shown as emanating from more or less the same distance from the tip, this is not necessary, in that a helix or spiral of apertures may alternative be used. As with this and all other embodiments disclosed herein, one or more of the ports may be used for non-electrode purposes, including an aesthetic administration.

1. Improved-coverage RF neurotomy instrumentation, comprising:
in introducer having a plurality of elongated, co-extensive cannula;
plurality of insulated, electrically conductive electrodes, one in each of the cannula;
each electrode having a proximal end configured for attachment to a source of energy and an exposed distal tip to deliver the energy to a localized region; and
wherein each electrode slides within its respective cannula so that the electrodes together enhance energy coverage area.

2. The improved-coverage RF neurotomy instrumentation of claim 1, wherein the distal tip of at least some of the electrodes is beveled to facilitate navigation.

3. The improved-coverage RF neurotomy instrumentation of claim 1, wherein at least some of the electrodes are constructed of a shape-memory material to control deployment.

4. The improved-coverage RF neurotomy instrumentation of claim 1, wherein the cannula are generally parallel and lie in the same plane.

5. The improved-coverage RF neurotomy instrumentation of claim 1, wherein the cannula are generally spoke-like in cross-section.

6. The improved-coverage RF neurotomy instrumentation of claim 1, wherein the introducer is curved.

7. The improved-coverage RF neurotomy instrumentation of claim 1, wherein at least one of the electrodes is radially deployed.

8. The improved-coverage RF neurotomy instrumentation of claim 1, wherein at least one side port for the administration of an anethestic.

9. The improved-coverage RF neurotomy instrumentation of claim 1, wherein at least some of the electrodes slide independently.

10. The improved-coverage RF neurotomy instrumentation of claim 1, wherein at least some of the electrodes slide in unison.
